(19) United States
(12) Patent Application Publication
(10) Pub. No.: US 2005/0214240 A1
(43) Pub. Date: Sep. 29, 2005

(54) Cosmetic preparation

(76) Inventors: Toshiyuki Ito, Tokyo (JP); Takashi Isshiki, Tokyo (JP); Junko Nakayama, Tokyo (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/508,547

(22) PCT Filed: Mar. 13, 2003
(86) PCT No.: PCT/JP03/02990

§ 371 Date: Sep. 28, 2004

(30) Foreign Application Priority Data
Mar. 29, 2002 (JP) 2002-95118
Sep. 26, 2002 (JP) 2002-281747

(51) Int. Cl. A61K 7/075; A61K 7/08

(52) U.S. Cl. 424/70.23

(57) ABSTRACT

A cosmetic preparation characterized by comprising (a) 0.01 to 10 wt % of a phospholipid, (b) 0.01 to 5 wt % of a nonionic surfactant having an HLB of 10 or higher and/or anionic surfactant, (c) 0.001 to 5 wt % of vitamin A or a derivative thereof, (d) 0.001 to 5 wt % of a stabilizer selected from among metal ion deactivators and antioxidants, and (g) water. The cosmetic preparation has satisfactory long-term stability, well spreads in application, gives excellent use feelings such as a fresh feeling and a nontacky feeling, and has high effects on the skin, such as a wrinkle diminishing effect.

### TECHNICAL FIELD

The present invention relates to a cosmetic preparation containing vitamin A and its derivatives, exhibiting excellent long-term stability, superior applicability, and good feeling during use.

### BACKGROUND ART

Vitamin A and its derivatives are known to be useful components effective for the prevention and medical treatment of diseases such as skin keratosis and for preventing skin aging, and are used in cosmetic compositions for the purpose of diminishing wrinkles. However, since vitamin A and its derivatives are structurally very unstable, being easily isomerized, decomposed, polymerized, or otherwise changed with light, air, heat, metal ions, and the like, adding these compounds to a cosmetic composition and maintaining them in a stable manner has been difficult.

For this reason, various types of stabilizers such as an antioxidant and a sequestering agent (a metal ion deactivator) are added to cosmetic compositions containing vitamin A or its derivatives to ensure storage stability (for example, Japanese Patent Applications Laid-open No. 04-210902, No. 06-032713). However, since sequestering agents known to be effective for stabilization of vitamin A and its derivatives are electrolytes, the sequestering agents adversely affect an emulsion system when added to an oil-in-water type emulsion cosmetic composition, causing the emulsion to agglomerate or associates, or creaming of the emulsion. Thus, it has been difficult to ensure sufficient long-term stability by the addition of a sequestering agent.

Although a method of adding a nonionic surfactant to stabilize emulsions has been known (e.g. Japanese Patent Applications Laid-open No. 11-228342 and No. 11-228377), this method is not satisfactory for securing long-term stabilization.

To ensure sufficient long-term stability, a method of adding a large amount of water-soluble polymers or adding higher alcohols has been employed. The addition of water-soluble polymers or higher alcohols increases the viscosity of the composition, whereby agglomeration, association, or creaming of emulsions can be prevented. However, the method has not been desirable from the viewpoint of adverse effects on the resulting compositions such as insufficient freshness, poor spreadability, and a sticky feel.

Therefore, development of a liquid cosmetic preparation containing vitamin A and its derivatives exhibiting superior long-term stability, excellently spreading during application, imparting excellent feeling of use such as freshness and a nonsticky sensation, and exhibiting a high effect on the skin such as a wrinkle diminishing effect has been desired.

### DISCLOSURE OF THE INVENTION

In view of this situation, the inventor of the present invention has conducted extensive studies to overcome the above-described drawbacks. As a result, the inventor has found that a cosmetic preparation comprising vitamin A and/or its derivative and water exhibiting superior long-term stability, excellently spreading during application, imparting excellent feeling of use such as freshness and a nonsticky sensation, and exhibiting a high effect on the skin such as a wrinkle diminishing effect can be obtained by using a conventionally known stabilizer such as a sequestering agent or an antioxidant in combination with a specific amount of phospholipids, a nonionic surfactant and/or anion surfactant having an HLB of 10 or higher.

Moreover, the inventor has found that a further combination of an oily component and a thickener can synergistically improve the effect of the cosmetic preparation on the skin. These findings have led to the completion of the present invention.

Specifically, the present invention provides a cosmetic preparation comprising the following components (a)-(g):
(a) 0.01 to 10 wt % of a phospholipid,
(b) 0.001 to 5 wt % of a nonionic surfactant having an HLB of 10 or higher and/or anionic surfactant,
(c) 0.001 to 5 wt % of vitamin A or a derivative thereof,
(d) 0.001 to 5 wt % of a stabilizer selected from among sequestering agents and antioxidants, and
(g) water.

### BEST MODE FOR CARRYING OUT THE INVENTION

The above-mentioned cosmetic preparation of the present invention, which comprises (a) a phospholipid, (b) a nonionic surfactant and/or anionic surfactant each having an HLB of 10 or higher, (c) vitamin A or a derivative thereof, (d) a stabilizer selected from among sequestering agents and antioxidants, and (g) water as essential components, can be broadly classified into two types of cosmetic preparations according to the required properties and the application.

Specifically, one is a cosmetic preparation comprising the following components:
(a) 0.01 to 5 wt % of a phospholipid,
(b) 0.01 to 5 wt % of a nonionic surfactant having an HLB of 10 or higher and/or anionic surfactant,
(c) 0.001 to 5 wt % of vitamin A or a derivative thereof,
(d) 0.001 to 5 wt % of a stabilizer selected from among sequestering agents and antioxidants, and
(g) water
   (hereinafter referred to as “first invention”); and the other is a cosmetic preparation comprising the following components:
(a) 0.1 to 10 wt % of a phospholipid,
(b) 0.001 to 5 wt % of a nonionic surfactant having an HLB of 10 or higher and/or anionic surfactant,
(c) 0.001 to 5 wt % of vitamin A or a derivative thereof,
(d) 0.001 to 5 wt % of a stabilizer selected from among sequestering agents and antioxidants,
(e) 1-50 wt % of an oily component,
(f) 0.001-10 wt % of a thickener, and
(g) water
   (hereinafter referred to as “second invention”).

Best modes for carrying out the first invention and the second invention will now be described.

The cosmetic preparation of the first invention comprises the above-mentioned components (a) to (d) and the component (g) as essential components.

The phospholipid used as the component (a) in the present invention promotes emulsification of oily components such as vitamin A or its derivatives or oil-soluble antioxidants and provides the cosmetic preparation with a moisturizing effect.

Any phospholipids commonly used in cosmetic compositions can be used as the component (a) in the present invention without specific limitation. As preferable specific examples, phosphatidyl choline, phosphatidyl ethanolamine, phosphatidyl serine, phosphatidyl glycerol, phosphatidyl inositol, sphyingolipid, and the like can be given.

As other components (a), analogues of the above phospholipids or compositions containing the above phospholipids and the like such as soybean lecithin, egg-yolk lecithin, or hydrogenated products of these lecithins can be given. These components (a) may be used either individually or in combination of two or more as appropriate.

The amount of the component (a) used in the cosmetic preparation of the first invention is 0.01-5 wt %, and to ensure more excellent long-term stability, 0.1-3 wt %. If the amount is less than 0.01 wt %, good long-term stability may not be obtained; if more than 5 wt %, the feeling on use may be impaired due to unfavorable stickiness and insufficient spreadability.

The nonionic surfactant having an HLB of 10 or higher and anionic surfactant are used as the component (b) of the present invention to increase dispersion stability of the component (a) and long-term stability of the cosmetic preparation and to promote feeling on use such as freshness.

In addition, the use of the component (b) in combination with the component (a) synergistically increases the long-term stability of the cosmetic preparations. Such a combined use particularly ensures excellent long-term stability of emulsified liquid cosmetic preparations which have been regarded as possessing poor stability.

Specific examples of the nonionic surfactant having an HLB of 10 or higher which can be used as the component (b) include:
polyoxyethylene glycerides such as polyoxyethylene (15) glyceryl monostearate and polyoxyethylene (15) glyceryl monooleate;
polyglycerol fatty acid esters such as hexaglyceryl monomyristate, decaglyceryl monolaurate, and decaglyceryl monostearate; polyoxyethylene sorbitan fatty acid esters such as polyoxyethylene (20) sorbitan monomyristate, polyoxyethylene (20) sorbitan monostearate, and polyoxyethylene (20) sorbitan trioleate; polyoxyethylene hydrogenated castor oil such as polyoxyethylene (20) hydrogenated castor oil and polyoxyethylene (60) hydrogenated castor oil; polyoxyethylene cholesteryl ethers such as polyoxyethylene (15) cholesteryl ether and polyoxyethylene (30) cholesteryl ether; polyoxyethylene phytosteryl ethers such as polyoxyethylene (10) phytosteryl ether and polyoxyethylene (20) phytosteryl ether; polyethylene glycol fatty acid esters such as polyethylene glycol (10E.O.) monolaurate and polyethylene glycol (25E.O.) monostearate;
polyoxyethylene alkyl ethers such as polyoxyethylene (9) lauryl ether, polyoxyethylene (7) cetyl ether, polyoxyethylene (20) stearyl ether; polyoxyethylene polyoxypropylene alkyl ethers such as polyoxyethylene (20) polyoxypropylene (4) cetyl ether and polyoxyethylene (30) polyoxypropylene (6) tetradecyl ether;
polyoxyethylene alkyl phenyl ethers such as polyoxyethylene (7.5) nonyl phenyl ether and polyoxyethylene (30) octylphenyl ether; and alkyl glucosides such as myristyl glucoside, cetostearyl glucoside, and cocoyl glucoside. These nonionic surfactants may be used either individually or in combination of two or more as appropriate. Of the above nonionic surfactants, polyoxyethylene hydrogenated castor oil and polyoxyethylene cholesteryl ether are preferable to obtain good long-term stability and an excellent feeling on use.

Specific examples of the anionic surfactant which can be used as the component (b) include:
salts of fatty acid such as sodium laurate, sodium myristate, sodium palmitate, and sodium isostearate; polyoxyethylene alkyl ether phosphates such as polyoxyethylene (10) lauryl ether phosphate, polyoxyethylene (5) cetyl ether phosphate, dipolyoxyethylene (10) cetyl ether phosphate; acyl taurates such as sodium methyl cocoyl taurate and sodium stearoyl methyl taurate; alkyl sulfates such as sodium lauryl sulfate and sodium cetyl sulfate; polyoxyethylene alkyl ether sulfates such as polyoxyethylene (2) lauryl ether sulfate and
polyoxyethylene (4) nonyl phenyl ether sulfate; N-acylamino acid salts; and N-acyl-N-alkylamino acid salts. These anionic surfactants may be used either individually or in combination of two or more as appropriate.

Of these components (b), phosphate surfactants or N-alkyl-N-alkyloyl taurates are preferable to obtain good long-term stability and an excellent feeling on use.

Polyoxyethylene alkyl ether phosphate surfactants such as polyoxyethylene alkyl ether phosphate are particularly preferable to improve dispersion stability of phospholipids and to obtain good long-term stability and an excellent feeling on use such as freshness.

The amount of the component (b) used in the cosmetic preparation of the first invention is 0.01-5 wt %, and to ensure more excellent long-term stability, 0.1-3 wt %. If the amount is less than 0.01 wt %, good long-term stability may not be obtained; if more than 5 wt %, the feeling on use may be impaired due to unfavorable stickiness and lack of freshness.

Vitamin A and its derivatives used as the component (c) in the present invention are effective for the prevention and medical treatment of diseases such as skin keratosis and for preventing skin aging and restoring aged skin. This component is added to the cosmetic compositions of the present invention for the purpose of diminishing wrinkles.

As the component (c), vitamin A and fatty acid esters of vitamin A such as vitamin A acetate and vitamin A palmitate can be given. These are preferably all-trans vitamin A or 13-cis vitamin A, or mixtures of these. The vitamin A and fatty acid esters of vitamin A may be used either individually or in combination of two or more as appropriate.

The amount of the component (c) used in the cosmetic preparation of the first invention is 0.001-5 wt %, and to ensure more excellent wrinkle diminishing effect, 0.01-3 wt %. If the amount is less than 0.001 wt %, a sufficient wrinkle diminishing effect may not be obtained; if more than 5 wt %, the feeling on use may be impaired due to an oiliness and lack of freshness.

The component (d) used in the present invention is a stabilizer. This component (d) increases the long-term stability of the component (c), vitamin A or its derivatives. There are no specific limitations to the component (c) inasmuch as this object can be achieved. As the sequestering agent among the stabilizers, alanine, edetate, sodium polyphosphate, sodium metaphosphate, phosphoric acid, and the like can be given.

As examples of oil-soluble antioxidants, dibutylhydroxytoluene, butylhydroxyanisol, α, β, γ, and δ-tocopherols, propylgallate, and L-ascorbic acid fatty acid ester can be mentioned. These compounds may be used either individually or in combination of two or more as appropriate.

As examples of water-soluble antioxidants, ascorbic acid and/or its salt, sulfites, hydrogensulfites, metahydrogensulfates, thiosulfites, thioglycerol, thiourea, thioglycolic acid, and cysteic acid salts can be mentioned. These water-soluble antioxidants may be used either individually or in combination of two or more as appropriate.

The amount of the component (d) used in the cosmetic preparation of the first invention is 0.001-5 wt %. A particularly preferable amount is 0.01-3 wt %, and to ensure more excellent long-term stability, 0.02-1 wt %. If the amount is less than 0.001 wt %, a sufficient long-term stability of vitamin A or its derivatives may not be obtained; if more than 5 wt %, the emulsion stability may be impaired.

The combined use of the sequestering agent and oil-soluble antioxidant is a preferred embodiment of the use of the component (d). In this instance, the total content of the both components is preferably 0.001-5 wt %, and particularly preferably 0.01-3 wt %.

In the first invention of the present invention, in addition to the above-mentioned components, water is added as an essential component (g) in an amount to provide an optimal solid content and optimal viscosity according to the object and application of the cosmetic preparation.

In the first invention of the present invention, in addition to the above-mentioned components, an oily component (component (e)) may be optionally added. The oily component has the function of promoting a moisturizing effect.

The compounds mentioned later in the description of the second invention can be given as the oily component used as the component (e). Preferable oily components are hydrocarbons, higher fatty acid esters, vegetable fats and oils, silicone oils, fluorine-containing oils, and sterols or their derivatives. Sterols are particularly preferred since, if used in combination with a phospholipid of the component (a), the sterols synergistically increase the long-term stability and promote the moisturizing effect.

Specific examples of the sterols and their derivatives include cholesterol, phytosterol, dihydrocholesterol, dihydrophytosterol, cholesteryl stearate, cholesteryl hydroxystearate, phytosteryl hydroxystearate, cholesteryl oleate, dihydrocholesteryl oleate, cholesteryl iso-stearate, dihydrocholesteryl iso-stearate, cholesteryl nonanoate, cholesteryl butyrate, dihydrocholesteryl butyrate, cholesteryl macadamia nut oil fatty acid, phytosteryl macadamia nut oil fatty acid, di(cholesteryl, behenyl, octyldodecyl) N-lauroyl-L-glutamate, di(cholesteryl, octyldodecyl)N-lauroyl-L-glutamate, di(phytosteryl behenyl, octyldodecyl)N-lauroyl-L-glutamate, and di(phytosteryl, octyldodecyl)N-lauroyl-L-glutamate. These sterols and sterol derivatives may be used either individually or in combination of two or more as appropriate.

The amount of the oily components used as the component (e) in the first invention is 0.001-20 wt %, and preferably 0.01-10 wt %. Although there are no specific limitations to the amount of sterols and sterol derivatives used as the component (e), an amount in the range of 0.001-5 wt % is preferable. A particularly preferable amount to ensure more excellent long-term stability is 0.01-3 wt %.

In addition to the above-described components, other components commonly used in cosmetic preparations, for example, polymers, aqueous components such as alcohols and polyols, fine particles, surfactants other than those mentioned above, UV absorbers, humectants, beauty ingredients, antiseptic agents, perfumes, refrigerants, and pH adjusting agents can be added to the extent that the effect of the first invention is not adversely affected.

The cosmetic preparation of the first invention prepared in this manner has satisfactory long-term stability of vitamin A which is the effective component, can excellently spread during application, gives excellent feeling of use such as a fresh feeling and a nonsticky sensation, and has high skin effect such as a wrinkle diminishing effect. In addition, incorporation of a phospholipid promotes emulsification of oily components such as vitamin A or its derivatives or oil-soluble antioxidants and increases the moisturizing effect.

Although the cosmetic preparation of the first invention may be either a solubilizing type or an emulsifying type, the invention is most suitably applied to a low viscosity liquid cosmetic preparation. The low viscosity liquid cosmetic preparation herein indicates a composition having a viscosity of less than 1000 mPa.s. The viscosity herein is a value determined using a Brookfield viscometer at 20° C. In the measurement, an appropriate rotor is selected from No. 1 to No. 4 rotors according to the viscosity of the cosmetic preparation. A single cylinder-type rotational viscometer (“Vismetron” manufactured by Shibaura Systems Co., Ltd.) can be given as a typical Brookfield viscometer.

Specific examples of particularly suitable applications are a face lotion and an essence lotion.

The cosmetic preparation of the second invention comprises the above-mentioned components (a) to (g) as essential components.

In the same manner as in the first invention, the phospholipid is added to the cosmetic preparation of the second invention as component (a) to promote emulsification of oily components such as vitamin A or its derivatives or oil-soluble antioxidants and to provide the cosmetic preparation with a moisturizing effect.

As the component (a), the same compounds as mentioned in the first invention can be used either individually or in combination of two or more as appropriate. The particularly preferable examples are also the same.

The amount of the component (a) used in the cosmetic preparation of the second invention is 0.1-10 wt %, and to ensure more excellent long-term stability, 0.1-5 wt %, with a particularly preferable amount being 0.2-3 wt %. If the amount is less than 0.1 wt %, good long-term stability may not be obtained; if more than 10 wt %, the feeling on use may be impaired due to unfavorable stickiness and insufficient spreadability.

The component (b) used in the second invention is added to increase dispersion stability of the phospholipid of the component (a), to improve long-term stability of emulsion cosmetic preparations, and to promote feeling during use such as freshness. The use of the component (b) in combination with the component (a) synergistically promotes the long-term stability.

Anionic surfactants are more preferable component (b) used in the second invention. The same anionic surfactants as mentioned in the first invention can be given as specific examples. Description of preferred specific examples of the anionic surfactants in the first invention also applies to the second invention. These anionic surfactants may be used either individually or in combination of two or more as appropriate.

The amount of the component (b) used in the cosmetic preparation of the second invention is 0.001-5 wt %, and to ensure more excellent long-term stability, 0.01-3 wt %, with a particularly preferable amount being 0.05-1 wt %. In the second invention, if the amount is less than 0.001 wt %, good long-term stability may not be obtained; if more than 5 wt %, the feeling on use may be impaired due to unfavorable stickiness and lack of freshness.

Vitamin A and its derivatives used as the component (c) in the second invention are added to achieve the same objectives as in the first invention, that is, prevention and medical treatment of diseases such as skin keratosis, prevention of skin aging, and restoration of aged skin, as well as diminishing wrinkles.

As the component (c), the same compounds as mentioned in the first invention can be used either individually or in combination of two or more as appropriate.

The amount of the component (c) used in the cosmetic preparation of the second invention is 0.001-5 wt %, and to ensure more excellent wrinkle diminishing effect, 0.01-3 wt %. If the amount is less than 0.001 wt %, a sufficient wrinkle diminishing effect may not be obtained; if more than 5 wt %, the feeling on use may be impaired due to an oiliness and lack of freshness.

In the same manner as in the first invention, the component (d) increases the long-term stability of the component (c), vitamin A or its derivatives, in the second invention.

In the same manner as in the first invention, there are no specific limitations to the component (d). Any sequestering agents, oil-soluble antioxidants, or water-soluble antioxidants can be used inasmuch as the above object can be achieved. The combined use of the sequestering agent, oil-soluble antioxidant, and water-soluble antioxidant is also preferable. Of these, the oil-soluble antioxidant is particularly preferable in the second invention. A particularly preferable combination is the combination of an oil-soluble antioxidant and a sequestering agent. A combination of an oil-soluble antioxidant and a water-soluble antioxidant is also preferable.

The same compounds described in the first invention can be given as specific preferable examples of the sequestering agent, oil-soluble antioxidant, and water-soluble antioxidant used in the second invention.

The amount of the component (d) used in the cosmetic preparation of the second invention is 0.001-5 wt %. A particularly preferable amount is 0.01-3 wt %, and to ensure more excellent long-term stability, 0.02-1 wt %. If the amount of the component (c) is less than 0.001 wt %, a sufficient long-term stability may not be obtained; if more than 5 wt %, the emulsion stability may be impaired.

When the oil-soluble antioxidant and sequestering agent are used in combination, the total content of both components is preferably 0.001-5 wt %, and particularly preferably 0.01-3 wt %. When the sequestering agent, oil-soluble antioxidant, and water-soluble antioxidant are used in combination as the component (d), the total content of all components is preferably 0.001-5 wt %, and particularly preferably 0.01-3 wt %.

In addition to the above-mentioned components, an oily component is added as a component (e) to the cosmetic preparation of the second invention. The component (e) is added to provide the skin with an emollient effect. An oily substance commonly used in cosmetics can be used. Any oils, irrespective of their origin (animal oils, vegetable oils, mineral oils, and synthetic oils) and their properties (solid oils, half-solid oils, liquid oils, and volatile oils), such as hydrocarbons, oils and fats, waxes, hydrogenated oils, ester oils, fatty acids, silicone oils, fluorine-containing oils, lanolin derivatives, oily gelling agents, and oil-soluble UV absorbers can be used.

Specific examples include hydrocarbons such as liquid paraffin, squalane, petroleum jelly, polyisobutylene, α-olefin oligomer, polybutene, paraffin wax, ceresin wax, micro-crystallin wax, montan wax, and Fischer-Tropsch wax; oils and fats such as olive oil, castor oil, mink oil, meadowfoam seed oil, avocado oil, macadamia-nut oil, and Japan wax;
waxes such as bees wax, carnauba wax, Candelilla wax, and spermaceti; esters such as cetyl 2-ethylhexanoate, isopropyl myristate, isopropyl palmitate, octyldodecyl myristate, glyceryl trioctanoate, oleyl oleate, isotridecyl iso-nonionate, polyglyceryl diisostearate, diglyceryl triisostearate, glyceryl tribehenate, pentaerythritol rosinate, diisostearyl malate, propylene glycol dicaprate, neopentyl glycol dioctanoate, cholesterol fatty acid ester, di(cholesteryl-behenyl-octyldodecyl)N-lauroyl-L-glutamate, and jojoba oil; fatty acids such as capric acid, lauric acid, myristic acid, palmitic acid, stearic acid, behenic acid, isostearic acid, oleic acid, linolic acid, linolenic acid, arachidonic acid, erucic acid, rosin acid, 2-ethylhexanoic acid, isopalmitic acid, lanolin fatty acid, adipic acid, and sebacic acid; liquid linear unsaturated aliphatic alcohols such as oleyl alcohol, linoleyl alcohol, and linolenyl alcohol; liquid branched aliphatic alcohols such as hexyldodecanol, isostearyl alcohol, and octyldodecanol;
silicones such as dimethylpolysiloxane, methylphenylpolysiloxane, decamethylcyclopentasiloxane, octamethylcyclotetrasiloxane, cross-linked organopolysiloxane, fluorine-modified silicone;
fluorine-containing oils such as a perfluorodecane, perfluorooctane, and perfluoropolyether; oily gelling agents such as lanolin, acetylated lanolin, isopropyl lanolin fatty acid, lanolin derivatives, dextrin fatty acid ester, sucrose fatty acid ester, starch fatty acid ester, 12-hydroxystearic acid, aluminum stearate, and calcium stearate; and oil-soluble UV absorbers such as p-ethylaminobenzoate, 2-ethylhexyl p-methoxycinnamate, 4-tert-butyl-4′-methoxydibenzoylmethane, oxybenzone can be given. These oils may be used either individually or in combination of two or more.

Among these oily components, ester oils, oils and fats, and the like are preferable to obtain an outstanding emollient effect. More preferable oily components are glyceryl trioctanate, propylene glycol dicaprate, oleyl oleate, isotridecyl isononanoate, jojoba oil, meadowfoam seed oil, and macadamia nut oil.

Sterols and their derivatives, as well as combinations of the sterol or a derivative with other oily components, can also be used as the component (e). By using sterols or their derivatives as the component (e) or a part of the component (e), the long-term stability and moisturizing effect can be improved as a result of the synergistic effect with the phospholipid component (a). The same compounds as described in the first invention can be given as specific preferable examples of the sterols and their derivatives used in the second invention.

The amount of the component (e) used in the cosmetic preparation of the second invention is 1-50 wt %, and to ensure more excellent emollient effect, 5-40 wt %. If the amount is less than 1 wt %, a sufficient emollient effect may not be obtained; if more than 50 wt %, the feeling on use may be impaired due to sticky sensation.

In addition to the above-mentioned components, a thickener as the component (f) is added to the cosmetic preparation of the second invention. The component (f) is added to provide the cosmetic preparation with a viscosity appropriate for use and particularly useful for providing an oil-in-water type emulsion cosmetic preparation with an appropriate viscosity.

There are no specific limitations to the component (f) inasmuch as the component can increase the viscosity of the emulsion cosmetic preparations. Water-soluble polymers, solid higher alcohols, and the like can be given as examples.

As examples of the water-soluble polymers, gum Arabic, tragacanth gum, Karaya gum, xanthan gum, tamarind gum, guar gum, hydroxypropyl guar gum, locust bean gum, quince seed, gellan gum, native gellan gum, polyacrylic acid, polymethacrylic acid, polyvinyl alcohol, polyethylene glycol, polyvinylpyrrolidone, polyacrylamide, polymethacrylamide, methacryloyloxyethyl trimethylammonium chloride, methacrylamide propyltrimethylammonium chloride, N-n-butoxymethyl acrylamide, N-isobutoxy acrylamide, tert-butylacrylamide sulfonate, dimethylaminopropyl methacrylamide, copolymers of these polymerizable monomer raw materials and the other polymerizable monomers, methylcellulose, hydroxyethylcellulose, hydroxypropylcellulose, carboxymethylcellulose, alginic acid, carageenan, hyaluronic acid, chondroitin sulfate, dermatan sulfate, collagen, chitin derivatives, pectin, gelatin, carboxyvinyl polymer, alkyl-modified carboxyvinyl polymer, and salts of these polymers can be given.

As specific examples of the solid higher alcohols, linear aliphatic saturated alcohols such as behenyl alcohol, lauryl alcohol, tridecyl alcohol, myristyl alcohol, pentadecyl alcohol, cetyl alcohol, heptadecyl alcohol, and stearyl alcohol; aliphatic higher alcohols such as cetostearyl alcohol and lanolin alcohol; and mixtures of these alcohols can be given. Alcohols having an aliphatic hydrocarbon chain with 12-22 carbon-atoms are preferable.

These water-soluble polymers and solid higher alcohols may be used either individually or in combination of two or more as appropriate.

Of these components (f), particularly preferred compounds are behenyl alcohol, cetostearyl alcohol, and cetyl alcohol among the higher alcohols, and xanthan gum, carboxyvinyl polymer, alkyl-modified carboxyvinyl polymer, and polyacrylamide among the water soluble polymers.

The amount of the component (f) used in the cosmetic preparation of the second invention is 0.001-10 wt %, and to provide an appropriate viscosity, an amount of 0.01-5 wt % is more preferable. If the amount is less than 0.001 wt %, a sufficient viscosity increasing effect may not be obtained; if more than 10 wt %, applicability may be impaired due to an excessively high viscosity and feeling of use may be impaired because of a sticky sensation.

The cosmetic preparation of the second invention is prepared by adding water of the component (g) to the components (a) to (f). The amount of the component (g) is suitably determined to provide a cosmetic preparation with an optimal solid content and optimal viscosity according to the object and application.

In addition to the above-described components, other components commonly used in cosmetic preparations, for example, polymers other than those mentioned above, aqueous components such as alcohols and polyols, fine particles, surfactants other than those mentioned above, UV absorbers, humectants, beauty ingredients, antiseptic agents, perfumes, refrigerants, and pH adjusting agents can be added to the cosmetic preparation of the second invention to the extent that the effect of the present invention is not adversely affected.

The cosmetic preparation of the second invention prepared in this manner has satisfactory long-term stability of vitamin A and the like which are the effective components, can excellently spread and does not drip during application, gives an excellent feeling of use such as a fresh, nonsticky sensation, emollient sensation, and has a high effect on the skin such as a wrinkle diminishing effect.

Although the cosmetic preparation of the second invention can be used for applications requiring a wide range of viscosity from high to low viscosities without any specific limitations, suitable applications are oil-in-water type emulsion cosmetic preparations and high viscosity cosmetic preparations. The high viscosity cosmetic preparation herein indicates a composition having a viscosity in the range of 1,000-1,000,000 mPa.s. The viscosity is measured using the same method as the method described in the first invention.

Specific applications include skin care cosmetic compositions such as a milky lotion, cream, essence, massage cosmetic composition, pack cosmetic composition, hand cream, body cream, and sunscreen cosmetic composition, and a foundation cosmetic composition for makeup. Directions of the use include application by hand, application using nonwoven fabric or the like impregnated with the cosmetic preparation, and the like.

### EXAMPLES

The present invention will be described in more detail with reference to Examples which should not be construed as limiting the present invention.

### Example 1

Liquid Cosmetic Preparation

Liquid cosmetic preparations were prepared from the components listed in Table 1 and Table 2 according to the following method of preparation. The cosmetic preparations obtained were evaluated for (1) applicability (spreadability), (2) feeling of use (freshness during use), (3) feeling of use (nonsticky sensation), (4) effect on the skin (wrinkle diminishing effect), and (5) long-term stability according to the following methods. Results are shown in Tables 1 and 2.

TABLE 1

| | | Preparation (values are wt %) | | | | | | | | | |
|---|---|---|---|---|---|---|---|---|---|---|---|
| Component | | 1 | 2 | 3 | 4 | 5 | 6 | 7 | 8 | 9 | 10 |
| 1 | Hydrogenated soybean lecithin | 0.01 | 0.1 | 3 | 5 | 3 | 1 | 1 | 1 | 3 | 1 |
| 2 | Cholesterol | — | — | — | — | — | — | 0.001 | 0.01 | 3 | 0.2 |
| 3 | Polyoxyethylene (60) hydrogenated castor | 0.01 | 0.1 | 3 | — | 2.5 | 1 | 0.1 | 1 | 3 | 1 |
| | oil | | | | | | | | | | |
| 4 | Sodium polyoxyethylene cetyl ether | — | — | — | 3 | 2.5 | — | — | — | — | 0.2 |
| | phosphate | | | | | | | | | | |
| 5 | Vitamin A palmitate | 0.001 | 0.01 | 3 | 5 | 3 | 1 | 0.01 | 1 | 1 | 1 |
| 6 | Dibutylhydroxytoluene | — | — | — | — | — | — | 0.001 | 0.01 | 0.95 | 0.5 |
| 7 | Isotridecyl isononanoate | 0.3 | 0.3 | 3 | — | — | 1 | 0.3 | 3 | 3 | 3 |
| 8 | Glycerol | 5 | 5 | 5 | 5 | 5 | 5 | 5 | 5 | 5 | 5 |
| 9 | Propylene glycol | 10 | 10 | 10 | 10 | 10 | 10 | 10 | 10 | 10 | 10 |
| 10 | Methylparabene | 0.3 | 0.3 | 0.3 | 0.3 | 0.3 | 0.3 | 0.3 | 0.3 | 0.3 | 0.3 |
| 11 | Perfume | 0.1 | 0.1 | 0.1 | 0.1 | 0.1 | 0.1 | 0.1 | 0.1 | 0.1 | 0.1 |
| 12 | Purified water | Balance | Balance | Balance | Balance | Balance | Balance | Balance | Balance | Balance | Balance |
| 13 | Disodium edetate | 0.001 | 0.01 | 1 | 0.1 | 0.1 | 5 | 0.05 | 0.05 | 0.05 | 0.05 |
| 14 | Disodium hydrogenphosphate | 0.14 | 0.14 | 0.14 | 0.14 | 0.14 | 0.14 | 0.14 | 0.14 | 0.14 | 0.14 |
| 15 | Sodium dihydrogenphosphate | 0.06 | 0.06 | 0.06 | 0.06 | 0.06 | 0.06 | 0.06 | 0.06 | 0.06 | 0.06 |

Evaluation item

| | | | | | | | | | | |
|---|---|---|---|---|---|---|---|---|---|---|
| (1) Applicability (spreadability) | ⊚ | ⊚ | ⊚ | ◯ | ⊚ | ⊚ | ⊚ | ⊚ | ⊚ | ⊚ |
| (2) Feeling of use (freshness during use) | ⊚ | ⊚ | ⊚ | ◯ | ◯ | ⊚ | ⊚ | ⊚ | ⊚ | ⊚ |
| (3) Feeling of use (nonsticky sensation) | ⊚ | ⊚ | ⊚ | ◯ | ◯ | ⊚ | ⊚ | ⊚ | ⊚ | ⊚ |
| (4) Effect on the skin (wrinkle diminishing effect) | ◯ | ⊚ | ⊚ | ⊚ | ⊚ | ⊚ | ⊚ | ⊚ | ⊚ | ⊚ |
| (5) Long-term stability (separation) | ◯ | ⊚ | ⊚ | ⊚ | ⊚ | ◯ | ⊚ | ⊚ | ⊚ | ⊚ |
| (6) Long-term stability (change in odor or color) | ◯ | ⊚ | ⊚ | ⊚ | ⊚ | ⊚ | ⊚ | ⊚ | ⊚ | ⊚ |

TABLE 2

| | | Preparation (values are wt %) | | | | | | | |
|---|---|---|---|---|---|---|---|---|---|
| Component | | 11 | 12 | 13 | 14 | 15 | 16 | 17 | 18 |
| 1 | Hydrogenated soybean lecithin | 1 | 1 | 0.001 | 8 | 1 | 1 | 1 | 1 |
| 2 | Cholesterol | — | — | — | — | — | — | — | — |
| 3 | Polyoxyethylene (60) hydrogenated castor | 1 | 1 | 1 | 1 | 0.001 | 8 | 1 | 1 |
| | oil | | | | | | | | |
| 4 | Sodium polyoxyethylene cetyl ether | — | — | — | — | — | — | — | — |
| | phosphate | | | | | | | | |
| 5 | Vitamin A palmitate | 0.0001 | 8 | 1 | 1 | 1 | 1 | 1 | 1 |
| 6 | Dibutylhydroxytoluene | — | — | — | — | — | — | — | — |
| 7 | Isotridecyl isononanoate | 3 | 3 | 3 | 3 | 3 | 3 | 3 | 3 |
| 8 | Glycerol | 5 | 5 | 5 | 5 | 5 | 5 | 5 | 5 |
| 9 | Propylene glycol | 10 | 10 | 10 | 10 | 10 | 10 | 10 | 10 |
| 10 | Methylparabene | 0.3 | 0.3 | 0.3 | 0.3 | 0.3 | 0.3 | 0.3 | 0.3 |
| 11 | Perfume | 0.1 | 0.1 | 0.1 | 0.1 | 0.1 | 0.1 | 0.1 | 0.1 |
| 12 | Purified water | Balance | Balance | Balance | Balance | Balance | Balance | Balance | Balance |
| 13 | Disodium edetate | 0.05 | 0.05 | 0.05 | 0.05 | 0.05 | 0.05 | 0.0001 | 8 |
| 14 | Disodium hydrogenphosphate | 0.14 | 0.14 | 0.14 | 0.14 | 0.14 | 0.14 | 0.14 | 0.14 |
| 15 | Sodium dihydrogenphosphate | 0.06 | 0.06 | 0.06 | 0.06 | 0.06 | 0.06 | 0.06 | 0.06 |

Evaluation item

| | | | | | | | | |
|---|---|---|---|---|---|---|---|---|
| (1) Applicability (spreadability) | ⊚ | Δ | ⊚ | X | ⊚ | X | ⊚ | ⊚ |
| (2) Feeling of use (freshness during use) | ⊚ | X | ⊚ | X | ⊚ | Δ | ⊚ | ⊚ |
| (3) Feeling of use (nonsticky sensation) | ⊚ | X | ⊚ | X | ⊚ | X | ⊚ | Δ |
| (4) Effect on the skin (wrinkle diminishing effect) | X | ⊚ | ⊚ | ⊚ | ⊚ | ⊚ | ⊚ | ⊚ |
| (5) Long-term stability (separation) | ⊚ | ⊚ | X | ⊚ | X | ⊚ | ⊚ | X |
| (6) Long-term stability (change in odor or color) | ⊚ | Δ | ⊚ | ⊚ | ⊚ | ⊚ | X | ⊚ |

(Method of Preparation)

A: Components (1)-(4) and (8)-(11) were heated and dissolved at 70° C.

B: Components (5)-(7) were heated and dissolved at 70° C., added to A, and mixed.

C: Components (12)-(15) were heated and dissolved at 70° C., added to B, and emulsified.

D: C was cooled to room temperature to obtain liquid cosmetic preparations.

(Evaluation of Applicability and Feeling of Use)

The cosmetic preparations 1-18 were evaluated for (1) applicability (spreadability), (2) feeling of use (freshness during use), (3) feeling of use (nonsticky sensation) by ten expert panelists according to the following (a) five-grade evaluation standard. The average of scores of each sample obtained by the five-grade evaluation standard was used for judgment according to the following (b) four-grade judgment standard.

(a) Five-grade evaluation standard

| (Score) | (Evaluation) |
|---|---|
| 4 | Excellent |
| 3 | Good |
| 2 | Fair |
| 1 | Slightly bad |
| 0 | Bad |

(b) Four-grade judgment standard

| (Score average) | (Judgment) |
|---|---|
| 3.5 or more | ⊚ |
| 2.5 or more and less than 3.5 | ◯ |
| 1.5 or more and less than 2.5 | Δ |
| Less than 1.5 | X |

(Evaluation Method of Effect on the Skin)

For the evaluation of (4) the effect on the skin (wrinkle diminishing effect), the cosmetic preparations 1-18 were applied to the skin of the ten expert panelists twice a day for one month, who evaluated the sample according to the following (c) four-grade evaluation standard. The resulting average of scores of each sample obtained was used for judgment according to the following (d) four-grade judgment standard.

(c) Four-grade evaluation standard

| (Score) | (Evaluation) |
|---|---|
| 4 | High wrinkle diminishing effect |
| 3 | Fair wrinkle diminishing effect |
| 2 | Slight wrinkle diminishing effect |
| 1 | No wrinkle diminishing effect |

(d) Four-grade judgment standard

| (Score average) | (Judgment) |
|---|---|
| 3.5 or more | ⊚ |
| 2.5 or more and less than 3.5 | ◯ |
| 1.5 or more and less than 2.5 | Δ |
| Less than 1.5 | X |

(Evaluation Method of Long-Term Stability)

The cosmetic preparations 1-18 were allowed to stand for one month in a thermostat chamber at 50° C. After storage, each sample was evaluated for (5) separation by appearance and (6) change in the odor and coloration over time. The results were judged according to the following (e) four-grade judgment standard.

(e) Four-grade judgment standard

| (Evaluation) | (Judgment) |
|---|---|
| No change | ⊚ |
| Slight change | ◯ |
| Some degree of change | Δ |
| Considerable degree of change | X |

(Results)

As is clear from Tables 1 and 2, the liquid cosmetic preparations of the present invention were excellent in respect of applicability (spreadability during use), feeling of use (freshness and nonsticky sensation during use), and effect on the skin (wrinkle diminishing effect). In addition, the components of the liquid cosmetic preparations of the present invention did not separate and these preparations exhibited no change in the odor and color after storage for one month in a thermostat chamber at 50° C. The cosmetic preparations were thus confirmed to have excellent long-term stability.

### Example 2

Face Lotion

A face lotion was prepared from the following components according to the following method.

| (Component) | (weight %) |
|---|---|
| 1. Hydrogenated soybean lecithin | 1.0 |
| 2. Phytosterol | 0.5 |
| 3. Sodium polyoxyethylene cetyl ether phosphate | 0.3 |
| 4. Glycerol | 10.0 |
| 5. Dipropylene glycol | 10.0 |
| 6. Vitamin A palmitate | 0.3 |
| 7. Natural vitamin E | 0.5 |
| 8. Purified water | Balance |
| 9. Disodium edetate | 0.2 |
| 10. Ethanol | 3.0 |
| 11. Phenoxy ethanol | 0.5 |
| 12. Methylparabene | 0.3 |
| 13. Xanthan gum | 0.01 |
| 14. Sodium dihydrogenphosphate | 0.2 |

(Method of Preparation)

A: Components (1)-(5) were heated and dissolved at 70° C.

B: Components (6) and (7) were heated and dissolved at 70° C., added to A, and mixed.

C: Components (8)-(14) were heated and dissolved at 70° C., added to B, and emulsified.

D: C was cooled to room temperature to obtain a face lotion.

The face lotion of Example 2 was excellent in long-term stability, applicability, feeling of use, and effect on the skin (wrinkle diminishing effect).

### Example 3

Face Lotion

A face lotion was prepared from the following components according to the following method.

| (Component) | (weight %) |
|---|---|
| 1. Hydrogenated soybean lecithin | 0.6 |
| 2. Phytosterol | 0.9 |
| 3. Sodium polyoxyethylene cetyl ether phosphate | 0.3 |
| 4. Glycerol | 5.0 |
| 5. Dipropylene glycol | 2.0 |
| 6. Vitamin A palmitate | 0.3 |
| 7. Ascorbil tetrahexyldecanoate | 1.0 |
| 8. Squalane | 8.0 |
| 9. Tocopherol acetate | 0.5 |
| 10. Purified water | Balance |
| 11. Disodium edetate | 0.2 |
| 12. Ethanol | 5.0 |
| 13. Polyethylene glycol 400 | 2.0 |

(Method of Preparation)

A: Components (1)-(5) were heated and dissolved at 70° C.

B: Components (6)-(9) were heated and dissolved at 70° C., added to A, and mixed.

C: Components (10)-(13) were heated and dissolved at 70° C., added to B, and emulsified.

D: C was cooled to room temperature to obtain a face lotion.

The face lotion of Example 3 was excellent in long-term stability, applicability, feeling of use, and effect on the skin (wrinkle diminishing effect).

### Example 4

Face Lotion

A face lotion was prepared from the following components according to the following method.

| (Component) | (weight %) |
|---|---|
| 1. Egg-yolk lecithin | 3.0 |
| 2. Cholesterol | 1.0 |
| 3. Polyoxyethylene (30) cholesteryl ether | 1.0 |
| 4. Glycerol | 10.0 |
| 5. Vitamin A palmitate | 3.0 |
| 6. Dibutylhydroxytoluene | 1.0 |
| 7. Dimethyl polysiloxane | 3.0 |
| 8. Glyceryl tri-2-ethylhexanoate | 5.0 |
| 9. Macadamia-nut oil | 1.0 |
| 10. Purified water | Balance |
| 11. Disodium edetate | 0.5 |
| 12. Diglycerine | 5.0 |

(Method of Preparation)

A: Components (1)-(4) were heated and dissolved at 70° C.

B: Components (5)-(9) were heated and dissolved at 70° C., added to A, and mixed.

C: Components (10)-(12) were heated and dissolved at 70° C., added to B, and emulsified.

D: C was cooled to room temperature to obtain a face lotion.

The face lotion obtained in Example 4 was excellent in long-term stability, applicability, feeling of use, and effect on the skin (wrinkle diminishing effect).

### Example 5

Face Lotion

A face lotion was prepared from the following components according to the following method.

| (Component) | (weight %) |
|---|---|
| 1. Hydrogenated soybean lecithin | 1.0 |
| 2. Cholesterol | 0.8 |
| 3. Isostearic acid | 0.5 |
| 4. Glycerol | 10.0 |
| 5. Vitamin A palmitate | 0.5 |
| 6. Dibutylhydroxytoluene | 0.1 |
| 7. Decamethylcyclopentasiloxane | 1.0 |
| 8. Oleyl oleate | 0.5 |
| 9. Isotridecyl isononanoate | 1.0 |
| 10. Perfume | 0.1 |
| 11. Purified water | Balance |
| 12. Disodium edetate | 0.2 |
| 13. Carboxyvinyl polymer | 0.001 |
| 14. Sodium hydroxide | 0.01 |

(Method of Preparation)

A: Components (1)-(4) were heated and dissolved at 70° C.

B: Components (5)-(10) were heated and dissolved at 70° C., added to A, and mixed.

C: Components (11)-(14) were heated and dissolved at 70° C., added to B, and emulsified.

D: C was cooled to room temperature to obtain a face lotion.

The face lotion of Example 5 was excellent in long-term stability, applicability, feeling of use, and effect on the skin (wrinkle diminishing effect).

### Example 6

### Face Lotion

A face lotion was prepared from the following components according to the following method.

| (Component) | (weight %) |
|---|---|
| 1. Hydrogenated soybean lecithin | 0.1 |
| 2. Cholesterol | 0.3 |
| 3. Sodium stearoyl methyltaurine | 0.05 |
| 4. Glycerol | 2.0 |
| 5. Propylene glycol | 1.0 |
| 6. Vitamin A palmitate | 0.05 |
| 7. Dibutylhydroxytoluene | 0.03 |
| 8. Methylphenyl polysiloxane | 0.05 |
| 9. 2-ethylhexyl paramethoxy cinnamate | 0.01 |
| 10. Purified water | Balance |
| 11. Disodium edetate | 0.2 |
| 12. Sodium citrate | 0.1 |

(Method of Preparation)

A: Components (1)-(5) were heated and dissolved at 70° C.

B: Components (6)-(9) were heated and dissolved at 70° C., added to A, and mixed.

C: Components (10)-(12) were heated and dissolved at 70° C., added to B, and emulsified.

D: C was cooled to room temperature to obtain a face lotion.

The face lotion of Example 6 was excellent in long-term stability, applicability, feeling of use, and effect on the skin (wrinkle diminishing effect).

### Example 7

Cream

Creams were prepared from the components listed in Table 3 and Table 4 according to the following method. The creams obtained were evaluated for (1) applicability (spreadability), (2) feeling of use (non-dripping), (3) feeling of use (freshness), (4) feeling of use (nonsticky sensation), (5) feeling of use (emollient sensation), (6) effect on the skin (wrinkle diminishing effect), (7) long-term stability (separation), and (8) long-term stability (color, odor) according to the following methods. The results are also shown in Tables 3 and 4.

TABLE 3

| | | Preparation (values are wt %) | | | | | | | | | | | | |
|---|---|---|---|---|---|---|---|---|---|---|---|---|---|---|
| Component | | 19 | 20 | 21 | 22 | 23 | 24 | 25 | 26 | 27 | 28 | 29 | 30 | 31 |
| 1 | Hydrogenated | 0.1 | 0.5 | 0.2 | 1.5 | 5 | 10 | 5 | 0.5 | 0.5 | 0.5 | 0.5 | 0.5 | 1.5 |
| | soybean | | | | | | | | | | | | | |
| | lecithin | | | | | | | | | | | | | |
| 2 | Cholesterol | — | — | — | — | — | — | — | 0.001 | 0.01 | 0.1 | 1 | 3 | 0.5 |
| 3 | Polyoxyethylene | — | — | — | — | — | — | — | — | — | — | 0.3 | 0.3 | 0.3 |
| | (60) hydrogenated | | | | | | | | | | | | | |
| | castor oil | | | | | | | | | | | | | |
| 4 | Sodium | 0.001 | 0.3 | 0.05 | 0.3 | 1 | 5 | 0.3 | 0.3 | 0.3 | 0.3 | 0.3 | 0.3 | 0.3 |
| | polyoxyethylene | | | | | | | | | | | | | |
| | (4) cetyl | | | | | | | | | | | | | |
| | ether phosphate | | | | | | | | | | | | | |
| 5 | Vitamin A palmitate | 0.001 | 0.01 | 0.01 | 0.3 | 3 | 5 | 0.3 | 1 | 1 | 1 | 1 | 1 | 0.3 |
| 6 | Dibutylhydroxytoluene | 0.001 | 0.01 | 0.01 | 0.1 | 1 | 5 | 0.1 | 0.001 | 0.001 | 0.001 | 0.001 | 0.001 | 0.1 |
| 7 | Cetanol | 3 | — | 3 | 3 | 5 | 10 | 3 | — | — | 1 | 1 | 1 | 3 |
| 8 | Isotridecyl | 0.5 | 1 | 1 | 1 | 15 | 5 | 20 | 1 | 1 | 1 | 1 | 1 | 1 |
| | isononanoate | | | | | | | | | | | | | |
| 9 | Glyceryl | 0.5 | 5 | 2 | 5 | 15 | 5 | 20 | 5 | 5 | 5 | 5 | 5 | 5 |
| | tri-2-ethylhexanoate | | | | | | | | | | | | | |
| 10 | Vaseline | — | 2 | 2 | 2 | 10 | 5 | 10 | 2 | 2 | 2 | 2 | 2 | 2 |
| 11 | Cholesteryl | — | — | — | 1 | — | — | — | — | — | 1 | 1 | 1 | 1 |
| | hydroxystearic | | | | | | | | | | | | | |
| | acid | | | | | | | | | | | | | |
| 12 | Glycerol | 5 | 5 | 5 | 5 | 5 | 5 | 5 | 5 | 5 | 5 | 5 | 5 | 5 |
| 13 | 1,3-Butylene glycol | 10 | 10 | 10 | 10 | 10 | 10 | 10 | 10 | 10 | 10 | 10 | 10 | 10 |
| 14 | Methylparabene | 0.2 | 0.2 | 0.2 | 0.2 | 0.2 | 0.2 | 0.2 | 0.2 | 0.2 | 0.2 | 0.2 | 0.2 | 0.2 |
| 15 | Perfume | 0.1 | 0.1 | 0.1 | 0.1 | 0.1 | 0.1 | 0.1 | 0.1 | 0.1 | 0.1 | 0.1 | 0.1 | 0.1 |
| 16 | Purified water | Bal- | Balance | Balance | Balance | Balance | Balance | Balance | Balance | Balance | Balance | Balance | Balance | Balance |
| | | ance | | | | | | | | | | | | |
| 17 | Disodium edetate | — | — | — | — | — | — | — | 0.001 | — | 0.01 | 0.1 | 3 | 0.1 |
| 18 | Magnesium | — | — | — | — | — | — | — | — | 0.001 | 0.01 | 0.1 | 3 | — |
| | ascorbic acid | | | | | | | | | | | | | |
| | phosphate | | | | | | | | | | | | | |
| 19 | Lactic acid | 0.03 | 0.03 | 0.03 | 0.03 | 0.03 | 0.03 | 0.03 | 0.03 | 0.03 | 0.03 | 0.03 | 0.03 | 0.03 |
| 20 | Sodium lactate | 0.2 | 0.2 | 0.2 | 0.2 | 0.2 | 0.2 | 0.2 | 0.2 | 0.2 | 0.2 | 0.2 | 0.2 | 0.2 |
| 21 | Xanthan gum | 0.5 | 0.001 | — | — | — | — | — | 0.001 | 0.001 | — | 0.1 | 0.2 | — |

Evaluation item

| | | | | | | | | | | | | | |
|---|---|---|---|---|---|---|---|---|---|---|---|---|---|
| (1) Applicability | ⊚ | ⊚ | ⊚ | ⊚ | ⊚ | ◯ | ◯ | ⊚ | ⊚ | ⊚ | ⊚ | ⊚ | ⊚ |
| (spreadability) | | | | | | | | | | | | | |
| (2) Applicability | ⊚ | ◯ | ⊚ | ⊚ | ⊚ | ⊚ | ⊚ | ◯ | ◯ | ⊚ | ⊚ | ⊚ | ⊚ |
| (non-dripping) | | | | | | | | | | | | | |
| (3) Feeling of use | ⊚ | ⊚ | ⊚ | ⊚ | ⊚ | ◯ | ◯ | ⊚ | ⊚ | ⊚ | ⊚ | ⊚ | ⊚ |
| (freshness during | | | | | | | | | | | | | |
| use) | | | | | | | | | | | | | |
| (4) Feeling of use | ⊚ | ⊚ | ⊚ | ⊚ | ⊚ | ◯ | ◯ | ⊚ | ⊚ | ⊚ | ⊚ | ⊚ | ⊚ |
| (nonsticky sensation) | | | | | | | | | | | | | |
| (5) Feeling of use | ◯ | ⊚ | ⊚ | ⊚ | ⊚ | ◯ | ⊚ | ⊚ | ⊚ | ⊚ | ⊚ | ⊚ | ⊚ |
| (emollient sensation) | | | | | | | | | | | | | |
| (6)Effect on the | ◯ | ⊚ | ⊚ | ⊚ | ⊚ | ⊚ | ⊚ | ⊚ | ⊚ | ⊚ | ⊚ | ⊚ | ⊚ |
| skin (wrinkle | | | | | | | | | | | | | |
| diminishing effect) | | | | | | | | | | | | | |
| (7) Long-term stability | ◯ | ◯ | ⊚ | ⊚ | ⊚ | ◯ | ◯ | ⊚ | ⊚ | ⊚ | ⊚ | ⊚ | ⊚ |
| (separation) | | | | | | | | | | | | | |
| (8) Long-term stability | ◯ | ⊚ | ⊚ | ⊚ | ⊚ | ◯ | ⊚ | ⊚ | ⊚ | ⊚ | ⊚ | ⊚ | ⊚ |
| (change in odor | | | | | | | | | | | | | |
| or color) | | | | | | | | | | | | | |

TABLE 4

| | | Preparation (values are wt %) | | | | | | | | | | | |
|---|---|---|---|---|---|---|---|---|---|---|---|---|---|
| Component | | 32 | 33 | 34 | 35 | 36 | 37 | 38 | 39 | 40 | 41 | 42 | 43 |
| 1 | Hydrogenated soybean | 1.5 | 1.5 | 1.5 | 1.5 | 0.05 | 15 | 1.5 | 1.5 | 1.5 | 1.5 | 1.5 | 1.5 |
| | lecithin | | | | | | | | | | | | |
| 2 | Cholesterol | — | — | — | — | — | — | — | — | — | — | — | — |
| 3 | Polyoxyethylene (60) | — | — | — | — | — | — | — | — | — | — | — | — |
| | hydrogenated castor oil | | | | | | | | | | | | |
| 4 | Sodium polyoxyethylene (4) | 0.3 | 0.3 | 0.0005 | 8 | 0.3 | 0.3 | 0.3 | 0.3 | 0.3 | 0.3 | 0.3 | 0.3 |
| | cetyl ether phosphate | | | | | | | | | | | | |
| 5 | Vitamin A palmitate | 0.0005 | 8 | 0.3 | 0.3 | 0.3 | 0.3 | 0.3 | 0.3 | 0.3 | 0.3 | 0.3 | 0.3 |
| 6 | Dibutylhydroxytoluene | 0.1 | 0.1 | 0.1 | 0.1 | 0.1 | 0.1 | 0.1 | 0.1 | 0.0005 | 8 | 0.1 | 0.1 |
| 7 | Cetanol | 3 | 3 | 3 | 3 | 3 | 3 | — | 15 | 3 | 3 | — | 3 |
| 8 | Isotridecyl isononanoate | 1 | 1 | 1 | 1 | 1 | 1 | 1 | 1 | 1 | 1 | 0.5 | 20 |
| 9 | Glyceryl | 5 | 5 | 5 | 5 | 5 | 5 | 5 | 5 | 5 | 5 | — | 15 |
| | tri-2-ethylhexanoate | | | | | | | | | | | | |
| 10 | Vaseline | 2 | 2 | 2 | 2 | 2 | 2 | 2 | 2 | 2 | 2 | — | 15 |
| 11 | Cholesteryl hydroxystearic | 1 | 1 | 1 | 1 | 1 | 1 | 1 | 1 | 1 | 1 | — | 15 |
| | acid | | | | | | | | | | | | |
| 12 | Glycerol | 5 | 5 | 5 | 5 | 5 | 5 | 5 | 5 | 5 | 5 | 5 | 5 |
| 13 | 1,3-Butylene glycol | 10 | 10 | 10 | 10 | 10 | 10 | 10 | 10 | 10 | 10 | 10 | 10 |
| 14 | Methylparabene | 0.2 | 0.2 | 0.2 | 0.2 | 0.2 | 0.2 | 0.2 | 0.2 | 0.2 | 0.2 | 0.2 | 0.2 |
| 15 | Perfume | 0.1 | 0.1 | 0.1 | 0.1 | 0.1 | 0.1 | 0.1 | 0.1 | 0.1 | 0.1 | 0.1 | 0.1 |
| 16 | Purified water | Balance | Balance | Balance | Balance | Balance | Balance | Balance | Balance | Balance | Balance | Balance | Balance |
| 17 | Disodium edetate | — | — | — | — | — | — | — | — | — | — | — | — |
| 18 | Magnesium ascorbic acid | — | — | — | — | — | — | — | — | — | — | — | — |
| | phosphate | | | | | | | | | | | | |
| 19 | Lactic acid | 0.03 | 0.03 | 0.03 | 0.03 | 0.03 | 0.03 | 0.03 | 0.03 | 0.03 | 0.03 | 0.03 | 0.03 |
| 20 | Sodium lactate | 0.2 | 0.2 | 0.2 | 0.2 | 0.2 | 0.2 | 0.2 | 0.2 | 0.2 | 0.2 | 0.2 | 0.2 |
| 21 | Xanthan gum | — | — | — | — | — | — | 0.0005 | — | — | — | 0.2 | — |

Evaluation item

| | | | | | | | | | | | | |
|---|---|---|---|---|---|---|---|---|---|---|---|---|
| (1) Applicability (spreadability) | ⊚ | ⊚ | ⊚ | X | ⊚ | X | ⊚ | X | ⊚ | X | ⊚ | ⊚ |
| (2) Applicability (non-dripping) | ⊚ | ⊚ | ⊚ | ⊚ | ⊚ | ⊚ | X | ⊚ | ⊚ | ⊚ | ⊚ | ⊚ |
| (3) Feeling of use (freshness during | ⊚ | Δ | ⊚ | X | ⊚ | X | ⊚ | Δ | ⊚ | Δ | ⊚ | X |
| use) | | | | | | | | | | | | |
| (4) Feeling of use | ⊚ | X | ⊚ | ⊚ | ⊚ | X | ⊚ | X | ⊚ | X | ⊚ | X |
| (nonsticky sensation) | | | | | | | | | | | | |
| (5) Feeling of use | ⊚ | ◯ | ⊚ | ⊚ | Δ | ⊚ | ⊚ | ⊚ | ⊚ | ⊚ | X | ⊚ |
| (emollient sensation) | | | | | | | | | | | | |
| (6) Effect on the skin (wrinkle | X | ⊚ | ◯ | ⊚ | ⊚ | ⊚ | ⊚ | ⊚ | Δ | ⊚ | ⊚ | ⊚ |
| diminishing effect) | | | | | | | | | | | | |
| (7) Long-term stability (separation) | ⊚ | ⊚ | X | ⊚ | X | ⊚ | X | ⊚ | ⊚ | ⊚ | ⊚ | X |
| (8) Long-term stability (change in | ⊚ | X | ⊚ | ⊚ | ⊚ | Δ | ⊚ | ⊚ | X | ⊚ | ⊚ | ⊚ |
| odor or color) | | | | | | | | | | | | |

(Method of Preparation)

A: Components (1)-(4), (12)-(14), (16), and (21) were heated and dissolved at 70° C.

B: Components (5)-(11) were heated and dissolved at 70° C., added to A, and emulsified.

C: Components (15) and (17)-(20) were added to B and mixed at 60° C.

D: C was cooled to room temperature to obtain a cream.

(Evaluation of Applicability and Feeling of Use)

Creams 19-43 were evaluated for (1) applicability (spreadability), (2) applicability (non-dripping), (3) feeling of use (freshness during use), (4) feeling of use (nonsticky sensation), and (5) feeling of use (emollient sensation) by ten expert panelists according to the following (a) five-grade evaluation standard. The average of scores of each sample obtained by the five-grade evaluation standard was used for judgment according to the following (b) four-grade judgment standard.

(a) Five-grade evaluation standard

| (Score) | (Evaluation) |
|---|---|
| 4 | Excellent |
| 3 | Good |
| 2 | Fair |
| 1 | Slightly bad |
| 0 | Bad |

(b) Four-grade judgment standard

| (Score average) | (Judgment) |
|---|---|
| 3.5 or more | ⊚ |
| 2.5 or more and less than 3.5 | ◯ |
| 1.5 or more and less than 2.5 | Δ |
| Less than 1.5 | X |

(Evaluation Method of Effect on the Skin)

For the evaluation of the wrinkle diminishing effect, the samples 19-43 were applied to the skin of the ten expert panelists twice a day for one month. The panelists evaluated the wrinkle diminishing effect of the samples according to the (c) four-grade evaluation standard. The average of scores of each sample obtained by the four-grade evaluation standard was used for judgment according to the four-grade judgment standard. The same four-grade evaluation standard and four-grade judgment standard used in Example 1 were employed.

(Evaluation Method of Long-Term Stability)

Long-term stability of the compositions 19-43 was evaluated by observing the external appearance for the presence or absence of separation and inspecting any change in odor and color in the same manner as in Example 1.

As is clear from Tables 3 and 4, the creams having the formulation according to the second invention were excellent in respect of applicability (spreadability during use, non-dripping), feeling of use (freshness, nonsticky sensation, emollient sensation during use), and effect on the skin (wrinkle diminishing effect) . In addition, after storage for one month in a thermostat chamber at 50° C., the components of the cosmetic preparations did not separate and these preparations exhibited no change in odor and color. The cosmetic preparations were thus confirmed to have excellent long-term stability.

### Example 8

Milky Lotion

A milky lotion was prepared from the following components according to the following method.

| (Component) | (weight %) |
|---|---|
| 1. Hydrogenated soybean lecithin | 1.0 |
| 2. Phytosterol | 0.5 |
| 3. Sodium polyoxyethylene (4) cetyl | 0.3 |
| ether phosphate | |
| 4. Glycerol | 5.0 |
| 5. Dipropylene glycol | 10.0 |
| 6. Vitamin A palmitate | 0.3 |
| 7. Natural vitamin E | 0.5 |
| 8. Propylene glycol dicaprate | 10.0 |
| 9. Behenyl alcohol | 0.5 |
| 10. Purified water | Balance |
| 11. Xanthan gum | 0.1 |
| 12. Disodium edetate | 0.2 |
| 13. Sodium dihydrogenphosphate | 0.2 |
| 14. Ethanol | 3.0 |
| 15. Phenoxy ethanol | 0.5 |
| 16. Methylparabene | 0.3 |

(Method of Preparation)

A: Components (l)-(5) were heated and dissolved at 70° C.

B: Components (6)-(9) were heated and dissolved at 70° C., added to A, and mixed.

C: Components (10)-(13) were heated and dissolved at 70° C., added to B, and emulsified.

D: C was cooled to room temperature, components (14)-(16) were added to obtain a milky lotion.

The milky lotion of Example 8 was excellent in long-term stability, applicability, feeling of use, and effect on the skin (wrinkle diminishing effect).

### Example 9

Massage Cosmetic Composition

A massage cosmetic composition was prepared from the following components according to the following method.

| (Component) | (weight %) |
|---|---|
| 1. Hydrogenated soybean lecithin | 1.5 |
| 2. Phytosterol | 0.9 |
| 3. Sodium stearoyl methyltaurine | 0.3 |
| 4. Glycerol | 5.0 |
| 5. Dipropylene glycol | 2.0 |
| 6. Sodium carboxymethylcellulose | 0.5 |
| 7. Purified water | Balance |
| 8. Disodium edetate | 0.2 |
| 9. Sodium hydrogensulfite | 0.1 |
| 10. Squalane | 10.0 |
| 11. Cetanol | 1.0 |
| 12. Vitamin A palmitate | 0.3 |
| 13. Ascorbil tetrahexyldecanoate | 1.0 |
| 14. Dibutylhydroxytoluene | 0.1 |
| 15. Ethanol | 5.0 |
| 16. Methylparabene | 0.2 |

(Method of Preparation)

A: Components (1)-(6) were heated and dissolved at 70° C.

B: Components (7)-(9) were heated at 70° C., added to A, and mixed.

C: Components (10)-(14) were heated and dissolved at 70° C., added to B, and emulsified.

D: C was cooled to room temperature, components (15)-(16) were added to obtain a massage cosmetic composition.

### Example 10

Cream

A cream was prepared from the following components according to the following method.

| (Component) | (weight %) |
|---|---|
| 1. Hydrogenated soybean lecithin | 2.0 |
| 2. Cholesterol | 1.0 |
| 3. Polyoxyethylene (60) hydrogenated | 0.5 |
| castor oil | |
| 4. Glycerol | 5.0 |
| 5. Dipropylene glycol | 10.0 |
| 6. Vitamin A palmitate | 0.3 |
| 7. Natural vitamin E | 0.5 |
| 8. Glyceryl tri-2-ethylhexanoate | 20.0 |
| 9. Behenyl alcohol | 1.0 |
| 10. Purified water | Balance |
| 11. Xanthan gum | 0.3 |
| 12. Disodium edetate | 0.2 |
| 13. Sodium dihydrogenphosphate | 0.2 |
| 14. Ethanol | 1.0 |
| 15. Phenoxy ethanol | 0.5 |
| 16. Methylparabene | 0.3 |

(Method of Preparation)

A: Components (1)-(5) were heated and dissolved at 70° C.

B: Components (6)-(9) were heated at 70° C., added to A, and mixed.

C: Components (10)-(13) were heated and dissolved at 70° C., added to B, and emulsified.

D: C was cooled to room temperature and components (14) -(16) were added to obtain a cream.

The cream of Example 10 was excellent in long-term stability, applicability, feeling of use, and effect on the skin (wrinkle diminishing effect).

### INDUSTRIAL APPLICABILITY

The cosmetic preparations of the present invention are excellent in applicability, feeling of use, and long-term stability. For example, the cosmetic preparation of the first invention is excellent in long-term stability, has outstanding effects of applicability (spreadability during use) and an excellent feeling of use (freshness and nonsticky sensation), and exhibits a superior effect on the skin (wrinkle diminishing effect).

The cosmetic preparation of the second invention exhibits, in addition to the above outstanding effects, excellent applicability (a non-dripping effect) and a superior feeling of use (an emollient sensation).

1. A cosmetic preparation comprising the following components:
(a) 0.01 to 10 wt % of a phospholipid,
(b) 0.001 to 5 wt % of a nonionic surfactant having an HLB of 10 or higher and/or anionic surfactant,
(c) 0.001 to 5 wt % of vitamin A or a derivative thereof,
(d) 0.001 to 5 wt % of a stabilizer selected from among sequestering agents and antioxidants, and
(g) water.

2. A cosmetic preparation comprising the following components:
(a) 0.01 to 5 wt % of a phospholipid,
(b) 0.01 to 5 wt % of a nonionic surfactant having an HLB of 10 or higher and/or anionic surfactant,
(c) 0.001 to 5 wt % of vitamin A or a derivative thereof,
(d) 0.001 to 5 wt % of a stabilizer selected from among sequestering agents and antioxidants, and
(g) water.

3. The cosmetic preparation according to claim 2, further comprising (e) 0.001-20 wt % of an oily component.

4. The cosmetic preparation according to claim 3, wherein the component (e) is a sterol or its derivative and added to the cosmetic preparation in an amount of 0.001-5 wt %.

5. The cosmetic preparation according to claim 1 or claim 2, wherein the cosmetic preparation is a low viscosity liquid preparation.

6. A cosmetic preparation comprising the following components:
(a) 0.1 to 10 wt % of a phospholipid,
(b) 0.001 to 5 wt % of a nonionic surfactant having an HLB of 10 or higher and/or anionic surfactant,
(c) 0.001 to 5 wt % of vitamin A or a derivative thereof,
(d) 0.001 to 5 wt % of a stabilizer selected from among sequestering agents and antioxidants,
(e) 1-50 wt % of an oily component,
(f) 0.001-10 wt % of a thickener, and
(g) water.

7. The cosmetic preparation according to claim 6, wherein the component (b) is an anionic surfactant.

8. The cosmetic preparation according to claim 6, wherein the component (b) is a phosphate surfactant or a N-alkyl-N-alkyloyl taurate.

9. The cosmetic preparation according to claim 6, wherein the component (b) is a polyoxyethylene alkyl ether phosphate surfactant.

10. The cosmetic preparation according to claim 6, wherein the component (d) is a mixture of a sequestering agent and an oil-soluble antioxidant.

11. The cosmetic preparation according to claim 6, wherein the component (d) is a mixture of a water-soluble antioxidant and an oil-soluble antioxidant.

12. The cosmetic preparation according to claim 6, wherein the component (d) is a mixture of a sequestering agent, water-soluble antioxidant, and an oil-soluble antioxidant.

13. The cosmetic preparation according to claim 6, wherein the component (e) is a sterol or its derivative.

14. The cosmetic preparation according to claim 6, wherein the component (f) is a water soluble polymer or a higher alcohol.

15. The cosmetic preparation according to claim 1 or claim 6, wherein the cosmetic preparation is an oil-in-water type emulsion cosmetic preparation.

16. The cosmetic preparation according to claim 1 or claim 6, wherein the cosmetic preparation is a high viscosity preparation.
